(19) United States
(12) Patent Application Publication
(10) Pub. No.: US 2005/0171596 A1
(43) Pub. Date: Aug. 4, 2005

(54) Stents with amphiphilic copolymer coatings

(76) Inventors: Joseph G. Furst, Lyndhurst, OH (US); William Brodbeck, South Euclid, OH (US)

Correspondence Address: Paul V. Keller, 4585 Liberty Rd., South Euclid, OH, 44121, US

(21) Appl. No.: 10/770,923

(22) Filed: Feb. 3, 2004

(51) Int. Cl. A61F 2/06

(52) U.S. Cl. 623/1.15; 623/1.42

(57) ABSTRACT

One aspect of the invention relates to a stent, a surface of which is coated with an amphiphilic copolymer that includes both hydrophobic and hydrophilic polymer chains. An amphiphilic copolymer coating according to the invention can serve as a carrier for a very broad range of drugs. The release rates of the drugs can be controlled, for example, through the length of the polymer chains, through their ratio, or through the degree of crosslinking.

### FIELD OF THE INVENTION

The present invention relates generally to the field of medicine and more particularly relates to drug coated stents.

### BACKGROUND OF THE INVENTION

Heart disease is still one of the most prevalent medical ailments in the world. Intraluminal endovascular stenting, a type of angioplasty procedure, is an alternative to conventional vascular surgery and is used to treat heart disease.

Several years ago, a product called a stent, named after Charles Stent, was introduced for use in angioplasty procedures. The stent reduced the angioplasty failure rate to about 15 percent. A stent is an expandable device that is generally mounted over an angioplasty balloon and deployed at the site of vascular narrowing. The balloon is inflated to expand the stent to physically open and return patency to the blood vessel. The balloon is removed, but the stent remains in the opened passageway. The first generation of expandable stents did not offer a controllable radial expansion. An improved stent disclosed in U.S. Pat. No. 4,733,665 overcame that limitation.

A common cause of failure after insertion of a stent into a body passageway is restenosis. Restenosis results from the body's reaction to the stent and the procedure for installing it. Restenosis can include closure of the stented passageway or, when caused by the stent procedure, closure of an adjacent passageway.

Restenosis can occur by at least three mechanisms: elastic recoil, negative arterial remodeling, and neointimal hyperplasia respectively involving the three layers of a blood vessel wall. Elastic recoil is the contraction of the outer vascular wall. Negative arterial remodeling is a shrinkage of the external elastic membrane. Neointimal hyperplasia is a thickening of the intimal layer.

Drugs can significantly inhibit or prevent the occurrence of restenosis, however, the continued need for the drugs after the stent has been inserted can require the patient to remain in a hospital for extended periods of time. Restenosis may occur days or weeks after the stent insertion procedure.

One approach to delivering restenosis inhibiting drugs is to provide them as a coating on a stent. U.S. Pat. No. 6,206,916 entitled “Coated Intraluminal Stent” discloses the use of a Trapidil coated stent to inhibit or prevent the occurrence of restenosis. U.S. Pat. No. 5,716,981 discloses the use of taxol or an analog or derivative thereof for use on a stent. U.S. Pat. Nos. 5,733,925 and 5,981,568 discloses taxol, a water soluble taxol derivative, cytochalasin, an analog thereof, or another type of cytoskeletal inhibitor, for use on a stent.

Several United States patents disclose the use of polymers to bind various drugs to the surface of a stent. Several of these polymers are disclosed in U.S. Pat. Nos. 5,578,075 and 5,679,400. U.S. Pat. No. 5,464,650 discloses a method of applying several coatings of a polymer that has been mixed with a drug to control the delivery of the drug in a body over a period of time.

At present, there are many biocompatible polymers. For example, poly(ethylene glycol) (PEG) is a water soluble polymer showing excellent biocompatibility and has been frequently used in biomedical applications. Similarly, polysiloxanes are widely used in the biomedical field and have been the subject of intense study both in the academic field as well as in industry.

Amphiphilic polymer networks have also been identified as potentially useful biomaterials. Amphiphilic polymer networks are co-continuous assemblages of hydrophilic and hydrophobic polymer chains that are able to swell in both hydrophilic solvents (e.g., water) and hydrophobic solvents (e.g., a liquid hydrocarbon). Because these materials swell in water, they generally fall into a class of compounds known as “hydrogels”.

The first amphiphilic membranes for biomaterials were developed over a decade ago. These were networks of hydrophilic polymers with the hydrophobic crosslinking agent, di-methacryl-telechelic polyisobutylene (MA-PIB-MA). Synthesis was accomplished by living carbocationic polymerization, which involves the free radical copolymerization and can use a variety of inexpensive, commercially available monomers, for example, N-dimethylaminoethyl methacrylate and dimethyl acrylamide.

Kennedy, U.S. Pat. No. 4,486,572 discloses the synthesis of styryl-telechelic polyisobutylene and amphiphilic networks comprising the copolymerization product of the styryl-telechelic polyisobutylene with vinyl acetate or N-vinyl-2-pyrollidone. Kennedy, U.S. Pat. No 4,942,204 discloses an amphiphilic copolymer network swellable in both water and n-heptane but insoluble in either, comprising the reaction product of an acrylate or methacrylate of a dialkylaminoalkyl with a hydrophobic bifunctional acryloyl or methacryloyl capped polyolefin. The preferred embodiment disclosed is an amphiphilic network having been synthesized by the free-radical copolymerization of a linear hydrophobic acrylate (A-PIB-A) or methacrylate capped polyisobutylene (MA-PIB-MA) with 2-(dimethylamino)ethyl methacrylate (DMAEMA). In a continuation-in-part to U.S. Pat. No. 4,942,204, Ivan et al. U.S. Pat. No. 5,073,381 discloses various amphiphilic copolymer networks that are swellable in water and n-heptane that comprise the reaction product of a hydrophobic linear acryloyl- or methacryloyl-capped polyolefin and a hydrophilic polyacrylate or polymethacrylate, such as N,N-dimethylacrylamide (DMAAm) and 2-hydroxyethylmethyl methacrylate (HEMA).

Hirt, U.S. Pat. No. 5,807,944 discloses a copolymer of controlled morphology comprising at least one oxygen permeable polymer segment and at least one ion permeable polymer segment, wherein the oxygen permeable segments and the ion permeable segments are linked together through a non-hydrolysable bond. The oxygen-permeable polymer segments are selected from polysiloxanes, perfluoroalkyl ethers, polysulfones, and other unsaturated polymers. The ion permeable polymers are selected from cyclic imino ethers, vinyl ethers, cyclic ethers, including epoxides, cyclic unsaturated ethers, N-substituted aziridines, beta-lactones, beta-lactanes, ketene acetates, vinyl acetates and phosphoranes.

U.S. application Ser. No. 09/433,660 discloses an amphiphilic network comprising the reaction product of hydrophobic crosslinking agents and hydrophilic monomers wherein the hydrophobic crosslinking agents are telechelic three-arm polyisobutylenes having acrylate or methacrylate end caps and wherein the hydrophilic monomers are acrylate or methacrylate derivatives.

### SUMMARY OF THE INVENTION

The following presents a simplified summary in order to provide a basic understanding of some aspects of the invention. This summary is not an extensive overview of the invention. It is intended neither to identify key or critical elements of the invention nor to delineate the scope of the invention. Rather, the primary purpose of this summary is to present some concepts of the invention in a simplified form as a prelude to the more detailed description that is presented later.

One aspect of the invention relates to a stent, a surface of which is coated with an amphiphilic copolymer that includes both hydrophobic and hydrophilic polymer chains. An amphiphilic copolymer coating according to the invention can reduce the effective surface area of a stent and can itself be relatively biologically inert. It can be flexible and stable under expansion of the stent. Significantly, it can serve as a carrier for a very broad range of drugs. The release rates of the drugs can be controlled, for example, through the length of the polymer chains, their ratio, or their degree of crosslinking.

Another aspect of the invention relates to a stent, a surface of which is coated with collagen containing a drug that inhibits stenosis, restenosis, or vascular narrowing. Collagen also exhibits many desirable properties for carrying certain drugs on stents.

Other aspects of the invention relate to manufacturing amphiphilic copolymer coated stents. One of these aspects relates to using a solvent to remove webbing of polymer that can form between stent struts and be a source of instability during stent expansion. Another of these aspects relates to polymerizing a solution containing monomers and a drug. A further of these aspects is a method of increasing the loading of a drug in an amphiphilic copolymer through multiple cycles of swelling the polymer with a solvent drug solution and evaporating the solvent.

Stents according to the invention are useful in treating vascular blockages. The delivery of drugs according to the invention can substantially reduce restenosis rates. In some cases, the effects of these treatments can be enhanced by oral or intravenous administration of the drugs.

A further aspect of the invention relates to treatments wherein microparticles, especially microparticles of amphiphilic copolymers, are used as carriers for drugs. For example, during angioplasty, drug-carrying amphiphilic copolymer microparticles can be suffused into injured vessel walls.

To the accomplishment of the foregoing and related ends, the following description and annexed drawings set forth in detail certain illustrative aspects and implementations of the invention. These are indicative of but a few of the various ways in which the principles of the invention may be employed. Other aspects, advantages and novel features of the invention will become apparent from the following detailed description of the invention when considered in conjunction with the drawings.

### BRIEF DESCRIPTION OF THE DRAWINGS

FIG. 1 is an illustration of an exemplary stent body.

FIG. 2 is an illustration of another exemplary stent body.

### DETAILED DESCRIPTION OF THE INVENTION

An exemplary stent according to the invention includes an expandable body suitable for dilating a blood vessel, an amphiphilic copolymer coating a surface of the body, and a drug carried by the polymer. The drug is of a type and is provided in an effective amount to significantly inhibit restenosis. In this context, inhibit means to slow the rate or reduce the occurrence of.

With regard to drug-coated stents, the drug is generally intended to inhibit the formation of a stenosis following installation of the stent. While the stent is presumably installed to treat an existing stenosis, a new stenosis would be of concern regardless of its location, mechanism of formation, or whether another stenosis existed previously. With this in mind, the term restenosis is used in this specification to encompass stenosis (formation of a stenosis where none previously existed) and vascular narrowing. Where these terms are separately listed, it is only for the sake of clarity to those who have not read this specification in detail.

A stent of the present invention provides a versatile platform for on-stent drug delivery. The amphiphilic block copolymer can be stable and flexible, whereby it retains its integrity during and after installation. The polymer can have a high degree of bio- and hemo-compatibility and can carry virtually any drug that might be of interest in connection with stents, including virtually any drug that is potentially useful in preventing restenosis. Release rates can be controlled as needed through variations in composition, loading, layering, and/or crosslinking.

While other stent/polymer combinations might have the features required for controlled release coatings of particular drugs on stents, stents according to the present invention have a significant advantage in versatility. Versatility is important in view of the need to conduct extensive testing prior to introducing any new material into the human body. A stent according to the present invention can easily, and with minimal testing, be adapted to implement advances in restenosis-preventing drug treatments.

An expandable stent is a stiff yet flexible substantially tubular stucture. It can be enlarged, typically under the pressure of an agioplasty baloon, from a first diameter to a second diameter. Preferably the enlargement in diameter occurs with little or no axial lengthening. Once enlarged, the stent resists shrinkage.

The expandable stent can have any suitable stucture. Examples include slotted tubes, coiled helical wires, coiled sheets, and heat-expandable tubes. The stent can maintain its expanded shape by any suitable means, including for example material memory or a ratcheting structure.

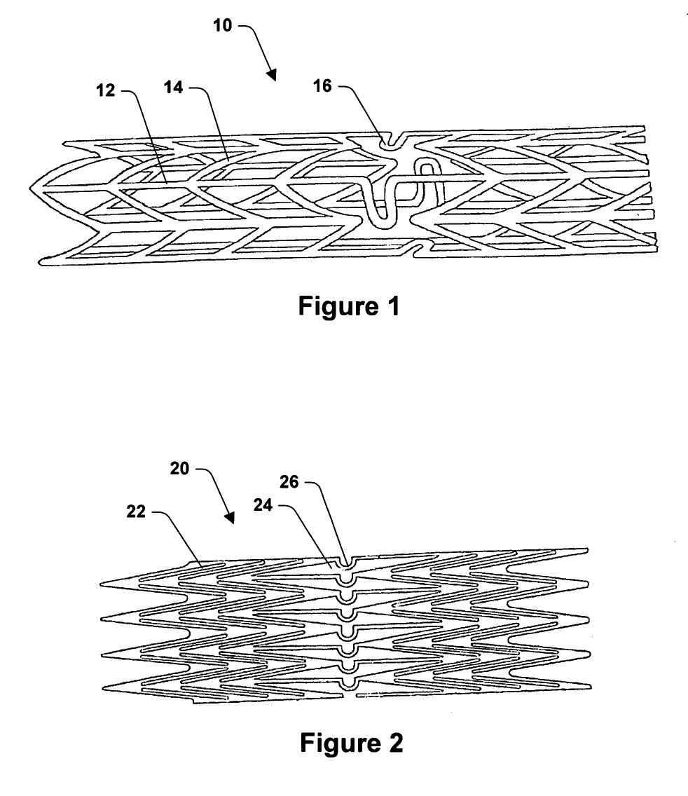

One stent structure is a tubular framework of axial and radial struts. FIG. 1 illustrates an exemplary tubular framework 10 of elongated members. Axial struts 12 substantially limit the axial lengthening and compression of the tubular framework 10. Radial struts 14, which connect axial struts 12, are formed at angles to the axial struts 12, whereby the distance between the axial struts 12 varies as the angles are expanded or contracted. U-shaped members 16 add flexibility to the stent along its axis. The shapes of the struts can widely vary.

FIG. 2 illustrates another exemplary tubular framework 20, this one in the form of a slotted tube. Narrow slots 22 divide the body 20 into regions that resemble the axial and radial struts of the tubular framework 10. Larger slots 24 create U-shaped members 26, adding flexibility to the stent 20 along its axis.

Preferably, the surfaces and edges of the stent are rounded, smoothed and/or blunted to minimize or prevent damage to body passageways as the stent is inserted and expanded. Rounding, smoothing or blunting can be accomplished mechanically, for example, by buffing, grinding, or sanding or by coating the stent, with a polymer for example.

The body of the stent can be of any suitable material, for example a metal such as stainless steel, tantalum, titanium, tungsten, gold, platium, iridium, rhodium, nitinol, or an alloy thereof, or an alloy of cobalt, nickel, chromium or molybdenum. The body can also be formed from a polymer such as poly(L-lactide), poly(D,L-lactide), poly(glycolide), poly(L-lactide-co-D,L-lactide), poly(L-lactide-co-glycolide), poly(D, L-lactide-co-glycolide), poly(glycolide-co-trimethylene carbonate), polydioxanone, polyethylene oxide, polycaprolactone, polyhydroxybutyrate, poly(phosphazene), poly(D,L-lactide-co-caprolactone), poly(glycolide-co-caprolactone), poly(phosphate ester), polyanhydrides, poly(ortho esters), poly(phoshate ester), poly(amino acid), polyacrylate, polyacrylamid, poly(hydroxyethyl methacrylate), elastin polypeptide co-polymer, polyurethane, polysiloxanes and their copolymers. Preferably the material is stainless steel. The material can be biostable or bioerodable.

The stent can be formed in any suitable manner. For example, the body can be initially formed into a tube or as a flat piece that is processed then rolled and joined at the edges. Processing can include, for example, stamping, laser cutting, laser ablation, die-cutting, chemical etching, plasma etching, or electromechanical. Where the material is rolled and joined, the edges can be connected together by a suitable method such as welding, soldering, brazing, adhesives, a lock and groove configuration, or a snap configuration. Microelectomechanical machining is especially useful for making small features such as grooves for a ratcheting mechanism.

Generally, the amphiphilic copolymer coating will provide biocompatibility, but in some cases, for example where the amphiphilic copolymer does not cover the entire surface, has slight instability, or has very large pores, it may be desireable to provide an underlying biocompatible coating. This coating may also serve to enhance binding of the amphiphilic copolymer coating, to provide smooth edges to the stent, or to provide a reservoir of the drug.

Any suitable biomechanical coating can be used. In one embodiment, the biocompatible coating includes a metal coating. The metal coating can be plated on at least a portion of the stent. The metal coating can include, for example, gold, platinum, titanium, nickel, tin, or a combination. In another embodiment, the biomechanical coating includes a polymer. The polymer can be, for example, polytetrafluoroethylene, polyethylene, poly(hydroxyethly methacrylate), poly(vinyl alcohol), polycaprolactone, poly(D, L-lactic acid), poly(L-lactic acid), poly(lactide-co-glycolide), poly(hydroxybutyrate), poly(hydroxybutyrate-co-valerate), polydioxanone, polyorthoester, polyanhydride, poly(glycolic acid), poly(glycolic acid-cotrimethylene cabonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, polyiminocarbonates, aliphatic polycarbonates, polyethylene oxide, polyethylene gylcol, poly(propylene oxide), polyacrylamides, polyacrylic acid (30-60% solution), polymethacrylic acid, poly(N-vinyl-2-pyrollidone), polyurethanes, poly(aminoacid), cellulosic polymers (e.g. sodium carboxymethyl cellulose, hydroxyethyl celluslose), collagen, carrageenan, alginate, starch, dextrin, gelatins, poly(lactide), poly(glycolide), polydioxanone, polycaprolactone, polyhydroxybutyrate, poly(phospazazene), poly(phosphate ester), poly(lactide-co-glycolide), poly(glycolide-co-trimethylene carbonate), poly(glycolide-co-caprolactone), polyanhydrides, polyamides, polyesters, polyethers, polyketones, polyether elastomers, parylene, polyether amide elastomers, polyacrylate-based elastomers, polyethylene, polypropylene, and/or and derivatives thereof.

One important class of stent materials and/or coatings forms a carpet-like surface. A carpet-like surface results when long chain molecules are bound at one end to the underlying stent surface. PTFE (including Teflon and Gortex), for example, can provide carpet-like surfaces. An amphiphilic block copolymer can fill or partially fill the interstices between long chain molecules and smooth over a carpet-like surface.

Various definitions of amphiphilic polymer are used in the literature. For purposes of the present disclosure, however, an amphiphilic polymer is a copolymer that includes both hydrophobic and hydrophilic polymer chains and is able to swell in both hydrophilic solvents (e.g., water) and hydrophobic solvents (e.g., n-heptane). This definition excludes, for example, a simple poly(ethylene glycol) polymer, which some have characterized as amphiphilic in view of its intermediate hydrophilicity.

Amphiphilic block copolymers include polymers having hydrophobic polymer chains crosslinked by hydrophilic polymer chains, polymers having hydrophilic polymer chains crosslinked by hydrophobic polymer chains, polymers having hydrophobic and hydrophilic polymer chains crosslinked by a crosslinking agent, and polymers in which multiple hydrophobic and hydrophilic chains are linked end to end. Amphiphilic graft copolymers include polymers having a hydrophilic backbone to which hydrophobic chains are attached and polymers having a hydrophobic backbone to which hydrophilic chains are attached. As the terms are used here, a graft copolymer is not, in general, a block copolymer. The assemblages of polymer chains are generally random.

Preferably, the amphiphilic copolymer is a block copolymer. Preferably, the polymer chains form a continuous network through either physical or chemical crosslinking. Physical crosslinking refers, for example, to bonding that occurs through aggregation of groups of hydrophobic segments, which results from their mutual attraction.

The monomers from which block copolymers are made generally include polymer chains. Under the terminology used here, these monomers may be referred to as macro-monomers. Likewise, the corresponding elements in the formed block copolymer can be referred to as macro-mers.

A hydrophobic polymer chain can be, for example, a polyolefin, preferably an olefin having 4 to about 12 carbon atoms as in poly(isobutylene), or a polysiloxane, such as poly(dimethylsiloxane). A hydrophilic polymer chain can be, for example, a poly(alkylene glycol), such as polyethylene glycol, a polyacrylate, such as polymers of methacrylate, 2-hydroxyethyl methylmethacrylate, or an aminoalkyl acrylate, such as N,N-dimethylacrylamide.

A preferred amphiphilic block copolymer network comprises macromolecular mers of polyethylene glycol (PEG), poly(isobutylene) (PIB), and poly(dimethylsiloxane) (PDMS). The polymer network can be synthesized by hydrosilation of allyl-terminated macromolecular monomers with pentamethylcyclopentasiloxane in toluene. The pore size of this network can be controlled by controlling the molecular weight of the hydrophilic macro-monomers. The strength can be controlled by the length of the hydrophobic macro-monomers and by the crosslink density. PDMS is oxyphilic and enhances transport of oxygen and related substances through the network.

More generally, macro-monomers, each a hydrophilic or hydrophobic polymer chain with functional end caps, can be polymerized together to form an amphiphilic block copolymer network. Suitable end caps include, for example, organic polyisocyanates, such as tolyene diisocyanate and diphenylmethane diisocyanate, acrylate, methacrylate and styryl groups. Block copolymers networks can also be generated by polymerizing polymer chains with monomers, for example, methacrylol capped PIB with dimethylaminoethyl methacrylate.

The solubility difference between hydrophobic and hydrophilic monomers can present difficulties during synthesis of amphiphilic block copolymers. One approach to overcoming this difficulty is to use a removable blocking agent to make a hydrophobic monomer temporarily hydrophilic or a hydrophilic monomer temporarily hydrophobic. For example a hydrophobic tertiary amine or amide can be made hydrophilic with a protonating blocking agent. For another example, a hydrophilic methacrylate can be made hydrophobic by the blocking agent trimethylsilyl chloride. The trimethylsilyl chloride can be removed by swelling the polymer in a 5% hydrochloric acid solution.

The amphiphilic block copolymer coating can be applied to the stent by any suitable means, including for example, spray coating, dip coating, spin coating, or brush coating. In some coating methods, webbing forms, for example in the angle between axial and radial struts of a stent. This webbing can crack during stent expansion. It is preferred to remove such webbing. Removal can be accomplish with a concentrated spray or stream of solvent.

Preferably, the stent is also coated with a material that is readily visible in vivo under fluoroscopic view. This coating is of value when guiding the stent into place within a body passageway.

The polymer carries a drug of a type, in a manner, and in an amount sufficient to significantly inhibit restenosis. The exemplary stent delays the onset of restenosis or reduces the occurrence of restenosis to a statically significant degree in comparison to an otherwise equivalent stent without the drug. Restenosis inhibiting drugs can be, for example, cytostatic or cytotoxic.

A stent according to the invention can be used to deliver virtually any drug, including without limitation, hydrophilic compounds, hydrophobic compounds, metal compounds, salts, polymers, antibodies, proteins, nucleic acids, and cells. It is further possible, with simple variations in the amphiphilic block copolymer composition, to control the release rate of any of these drugs.

Diverse drugs are of interest in connection with preventing restenosis, including the following:
anticoagulants, including heparin, low molecular weight herapins, hirudin, warfarin, bivalirudin, and Vasoflux;
antithrombotic agents, including argatroban, efegatran, tick anticoagulant peptide, Ppack, HMG-COA reductase inhibitors, thromboxane A2 receptor inhibitors, endothelium-derived relaxing factor plasminogen activator inhibitor, tissue-type plasminogen activator (tPA), ReoPro, fibrin and fibrin peptide A, chrysalin, D-Phe-ProArg chloromethyl ketone, and glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban)
antiplatelet agents, including aspirin, dipyridamole, apo-dipyridamole, persantine, prostacyclin, ticlopidine, clopidogrel, cromafiban, and cilostazol;
antiproliferative agents, including triazolopyrimidine (Trapidil), paclitaxel (Taxol), tranilast (Rizaben), Rapamycin (sirolimus), tacrolimus, angiopeptin, butyrate, ceramide, ciprostene, cultrazine, cyclosporine, EGF-genistein, fucoidans, halofuginone, lioprost, ketaserine, predisone, dipyridamole, 17-beta-estradiol, suramin, nitric oxide donors (including FK409, linsidomine, and molsidomine), phytoestrogens, colchine, probucol, terbinafine, etoposide, doxorubicine, beraprost sodium, Resten-NG, actinomycin D, phosphorylcholine, Batimastat, and calcium channel blockers (including, amlodipine, verapamil, diltiazem HCL, and nifedipine);
anti-inflammatory agents, including dipyridamole, and glucocorticoids (including betamethazone, rosiglitazone, and dexamethazone);
lipid-lowering drugs, including omega-3 fatty acids, prostaglandin I₂, prostaglandin E1, pravastatin, lovastatin, cerivastatin, fluvastatin, and simvastatin;
specific growth factor antagonists, including lanreotide;
antioxidants, including alpha-tocopherol, beta-carotene, and probucol;
genetic materials, including those carried by viral vectors, plasmids, and lipid-based carriers (including, antisense oligonucleotides such as AVI-2221, INX-3280, RestenASE), ribosymes, and cytochalasin B;
angiogenic growth factors, including platelet derived growth factors alpha and beta;
antihypertension drugs, including angiotensin converting enzyme inhibitors and angiotensin II receptor antagonists (including captopril, quinapril, cilazapril, losartan, and valsartan)
radioactive compounds, including metal salts;
lymphokines including (IL)-1, -2, -3, and -4, as well as colony stimulating factors such as G-CSF, GM-CSF, and M-CSF.
   Most of these drugs have analogs and derivative that are also of interest in preventing restenosis. Analogs and derivatives include minor alterations in structure and substitutions or additions of atoms or functional groups that do not alter, except perhaps by degree, the primary mechanism of action. For example paclitaxel derivatives include, without limitation, taxotere, baccatin, 10-deacetyltaxol, 7-xylosyl-10-deacetyltaxol, cephalomannine, 10-deacetyl-7-epitaxol, 7 epitaxol, 10-deacetylbaccatin III, and 10-deacetylcephaolmannine.

Virtually any of the drugs of interest in preventing restenosis can be delivered using an amphiphilic block copolymer on a stent according to the present invention. A preferred stent/polymer combination can deliver many of these drugs with little or no variation in the polymer composition. For example, an amphiphilic block copolymer networks comprising PEG, PIB, and PDMS can be used to deliver with a controlled release rate any of triazolopyrimidine, paclitaxol, and sirolimus on the one hand and any of stem cells, antibodies, genetic materials, and lymphokines on the other. Where some variation is required to achieve appropriate release rates for these various drugs or drug groups, it is preferred that these variations be limited to the ratios and/or chain lengths of the macro-monomers and the degree of crosslinking.

The polymer can be loaded with the drug by any suitable means. One approach is to include the drug with the macro-monomers as they are polymerized together. Another is to dissolve the drug in a solvent and swell the polymer with the solvent. All or part of the solvent can be evaporated and the polymer swelled again to increase the drug loading level.

The drug can remain in the stent, as when the drug is a radiation source. More generally, however, it is preferred that the drug be released by the stent, either to be absorbed in the vessel wall around the stent or to be released downstream. In one embodiment, the drug is of a type that can absorb and be stored in vessel walls.

An advantage of the present invention is that amphiphilic copolymers can be tailored to provide virtually any desired release rate. Non-soluble amphiphilic block copolymers generally provide release rate kinetics in the range from about 0.4 order to about first order. Within this framework, a particular release rate may be targeted. In one embodiment, the stent can release from about 10 to about 90 percent of the drug within the first thirty days of installation, preferably from about 20 to about 60 percent of the drug within the first thirty days. In another embodiment, the sent releases from about 10 to about 90 percent of the drug within the first six hours of installation, preferably from about 20 to about 60 percent of the drug within the first six hours.

In one embodiment of the invention, two surface of the stent have two different drug/polymer combinations and release drugs at two different rates. For example, surfaces near the ends of the stent can be coated with a polymer carrying a first drug that acts locally near these ends, whereas the center can be coated with another polymer carrying a second drug that releases into the blood and acts over a greater area. In another embodiment, the polymer is provided in multiple layers, each having a different drug/polymer combination or a different drug loading level.

A variety of options are available for controlling the release rate. The release rate can be varied though any of: the identity of the macro-monomers, the lengths of the macro-monomer chains, the ratios of the macro-monomers, the degree of crosslinking in the copolymer network, the loading of the drug, and the thickness of the amphiphilic copolymer coating. Additional release patterns can be obtained by employing multi-layer coatings, which may include layers that are not amphiphilic copolymers. For example, a barrier layer may be formed over the amphiphilic copolymer to slow the release rate. One of the biocompatible coatings listed above would be appopriate for a barrier coating. A preferred barrier layer comprises parylene or a derivative thereof.

Local drug delivery through a stent coating often allows the use of higher drug concentrations in those locations where the drug is needed than could safely be achieved with system wide delivery. Nonetheless, there can be synergy between stent-based delivery and system-wide delivery. Thus, in one embodiment, treatment with a drug-coated stent according to the invention is combined with oral or intravenous dosage of the same drug.

Amphiphilic copolymers have unique advantages for drug-coated stents. Nonetheless, in certain situations, collagen can be used as an alternative. As a stent coating, collagen can smooth over surfaces and create bio- and hemo-compatibility. In one embodiment, a stent with a carpet like surface is coated with collagen. Preferably, the collagen contains a drug of a type and in an amount to significantly inhibit restenosis. In one embodiment, the collagen contains either stem cells, antibodies, genetic materials, or lymphokines in an amount to significantly inhibit thrombosis and/or stenosis. Of particular interest in this group are stem cells and GM-CSF. In another embodiment, the drug is triazolopyrimidine a derivative thereof, or an analog thereof.

In addition to a stent coating, an amphiphilic copolymer network can be used to form microparticles. Such microparticles can also carry and deliver at a controlled rate a wide range of drugs. Microparticles have a size range from about 10 nanometers to about 200 micrometers, preferably from about 50 nm to about 1 micrometer.

One aspect of the invention relates to the use of microparticles, especially amphiphilic copolymer microparticles, to carry a drug that inhibits restenosis. During angioplasty, after the vessel has been expanded by filling an inner balloon, the particles can be injected into a manifold between the inner balloon and a porous outer balloon. The particles escape through the pores and suffuse into the damaged tissue surrounding the balloon. There, the particles release the drug where it is most needed.

The invention has been shown and described with respect to certain aspects, examples, and embodiments. While a particular feature of the invention may have been disclosed with respect to only one of several aspects, examples, or embodiments, the feature may be combined with one or more other features of the other aspects, examples, or embodiments as may be advantageous for any given or particular application. Furthermore, to the extent that the term “includes” is used in either the detailed description or the claims, the term is intended to be inclusive in the manner of the term “comprising.”

1. A stent, comprising:
an expandable body suitable for dilating a blood vessel; and
an amphiphilic copolymer coating a surface of the body;
wherein the amphiphilic copolymer comprises a continuous network including both hydrophobic and hydrophilic polymer chains that is able to swell in both hydrophobic and hydrophilic solvents.

2. The stent of claim 1, wherein the copolymer is an amphiphilic block copolymer.

3. The stent of claim 1, wherein the amphiphilic block copolymer coating carries a drug of a type and in an effective amount to significantly inhibit one or more of stenosis, restenosis, and vascular narrowing.

4. The stent of claim 2, wherein the amphiphilic block copolymer coating carries a drug of a type and in an effective amount to significantly inhibit one or more of stenosis, restenosis, and vascular narrowing.

5. The stent of claim 4, wherein the amphiphilic block copolymer comprises poly(alkylene glycol) chains and poly(olefin) chains.

6. The stent of claim 4, wherein the block copolymer further comprises polysiloxane chains.

7. The stent of claim 7, wherein the drug is selected from the group consisting of triazolopyrimidine, paclitaxol, sirolimus, derivatives thereof, and analogs thereof.

8. The stent of claim 7, wherein the drug is triazolopyrimidine, a derivative thereof, or an analog thereof.

9. The stent of claim 4, wherein the drug is selected from the group consisting of stem cells, antibodies, genetic materials, and lymphokines.

10. The stent of claim 9, wherein the drug is stem cells.

11. The stent of claim 9, wherein the drug is GM-CSF.

12. The stent of claim 4, wherein the copolymer can be designed to carry any of the drugs triazolopyrimidine, paclitaxol, and sirolimus and release from about 10 to about 90 percent of the drug within the first thirty days of installation in an artery of a living human being by varying lengths of the hydrophobic and hydrophilic polymer chains, ratios between chains, and/or extent of cross-linking.

13. The stent of claim 4, wherein the copolymer can be designed to carry any of the drugs stem cells, antibodies, genetic materials, and lymphokines and release from about 10 to about 90 percent of the drug within the first thirty days of installation in an artery of a living human being by varying lengths of the hydrophobic and hydrophilic polymer chains, ratios between chains, and/or extent of cross-linking.

14. The stent of claim 4, wherein the stent is coated with a plurality of layers, wherein one of the layers acts as a barrier to diffusion of the drug.

15. The stent of claim 14, wherein the barrier layer comprises parylene or a derivative thereof.

16. The stent of claim 4, wherein the stent once installed in an artery of a living human being releases from about 10 to about 90 percent of the drug within the first thirty days of installation.

17. The stent of claim 4, wherein the stent once installed in an artery of a living human being releases from about 10 to about 90 percent of the drug within the first six hours of installation.

18. The stent of claim 1, wherein the surface comprises polymer chains bound at one end to form a carpet-like structure and the amphiphilic polymer at least partially fills interstices within the carpet-like structure.

19. The stent of claim 4, wherein the surface comprises polymer chains bound at one end to form a carpet-like structure and the amphiphilic polymer at least partially fills interstices within the carpet-like structure.

20. The stent of claim 4, wherein the polymer coating remains stable under expansion of the body.

21. The stent of claim 1, wherein the polymer is bioerodable.

22. The stent of claim 4, wherein the polymer is bioerodable.

23. The stent of claim 1, wherein the polymer is biostable.

24. The stent of claim 4, wherein the polymer is biostable.

25. A stent, comprising:
an expandable body suitable for dilating a blood vessel, the body having a surface to which polymer chains are bound at one end to form a carpet-like structure;
collagen coating the polymer-chain covered surface and at least partially filling interstices within carpet-like structure; and
within the collagen, a drug selected from the group consisting of triazolopyrimidine, a derivative thereof, or an analog thereof, stem cells, antibodies, genetic materials, and lymphokines in an amount effective to significantly inhibit one or more of stenosis, restenosis, and vascular narrowing.

26. The stent of claim 25, wherein the drug is triazolopyrimidine a derivative thereof, or an analog thereof.

27. The stent of claim 25, wherein the drug is selected from the group consisting of stem cells, antibodies, genetic materials, and lymphokines.

28. The stent of claim 27, wherein the drug is stem cells.

29. The stent of claim 27, wherein the drug is GM-CSF.

30. A method of manufacturing a stent, comprising:
providing an expandable body suitable for dilating a blood vessel; and
forming over a surface of the body a coating comprising an amphiphilic block copolymer, wherein the amphiphilic block copolymer comprises a network of both hydrophobic and hydrophilic polymer chains that is able to swell in both hydrophobic and hydrophilic solvents.

31. The method of claim 30, further comprising:
forming a solution comprising a solvent and a drug that inhibits one or more of stenosis, restenosis, and vascular narrowing; and
swelling the polymer with the solution.

32. The method of claim 31, further comprising:
evaporating to remove at least some of the solvent from the polymer; and
swelling the polymer a second time with the same or another solution containing the drug.

33. The method of claim 30, wherein, forming a coating comprising an amphiphilic block copolymer comprises coating the surface with a solution of macro-monomers together with a drug and polymerizing the macro-monomers.

34. The method of claim 30, wherein the copolymer can be designed to carry any of the drugs triazolopyrimidine, paclitaxol, and sirolimus and release from about 10 to about 90 percent of the drug within the first thirty days of installation in an artery of a living human being by varying lengths of the hydrophobic and hydrophilic polymer chains, ratios between chains, and/or extent of crosslinking.

35. The method of claim 30 wherein the copolymer can be designed to carry any of the drugs stem cells, antibodies, genetic materials, and lymphokines and release from about 10 to about 90 percent of the drug within the first thirty days of installation in an artery of a living human being by varying lengths of the hydrophobic and hydrophilic polymer chains, ratios between chains, and/or extent of crosslinking.

36. The method of claim 30, further comprising forming the stent by a process comprising microelectomechanical machining.

37. The method of claim 36, wherein the microelectromechanical machining is used to form teeth or other indentations that are part of a ratcheting mechanism.

38. The method of claim 30, wherein the body comprises a web-like structure and the polymer forms webbing within openings in the structure, the method further comprising applying a concentrated stream or spray of solvent to remove the webbing.

39. A method of treating an occluded blood vessel in a living human being, comprising,
inserting an expandable stent according to claim 1 within the vessel; and
expanding the stent.

40. The method of claim 38, wherein the amphiphilic block copolymer carries a drug of a type and in an effective amount to significantly inhibit one or more of stenosis, restenosis, and vascular narrowing.

41. The method of claim 40, wherein the drug is selected from the group consisting of triazolopyrimidine, paclitaxol, sirolimus, lymphokines, derivatives thereof, and analogs thereof.

42. The method of claim 40, wherein the drug is selected from the group consisting of stem cells, antibodies, genetic materials, and lymphokines.

43. The method of claim 42, wherein the drug is stem cells.

44. The method of claim 42, wherein the drug is GM-CSF.

45. The method of claim 40, further comprising administering the drug to the patient either orally or intravenously.

46. A method of treating an occluded blood vessel in a living human being, comprising,
inserting an expandable stent within the vessel; and
expanding the stent with a two layer balloon, the inner layer being relatively impermeable and the outer layer being relatively permeable; and
injecting microparticles containing a drug between the two layers, whereby the microparticles are distributed through the outer layer of the balloon.

47. The method of claim 46, wherein the microparticles comprises an amphiphilic block copolymer, the amphiphilic block copolymer comprising a network including both hydrophobic and hydrophilic polymer chains that is able to swell in both hydrophobic and hydrophilic solvents.

48. The method of claim 47, wherein the amphiphilic block copolymer coating carries a drug of a type and in an effective amount to significantly inhibit one or more of stenosis, restenosis, and vascular narrowing.

49. The stent of claim 48, wherein the drug is selected from the group consisting of triazolopyrimidine, paclitaxol, sirolimus, derivatives thereof, and analogs thereof.

50. The stent of claim 48, wherein the drug is selected from the group consisting of stem cells, antibodies, genetic materials, and lymphokines.

51. The stent of claim 50, wherein the drug is stem cells.

52. The stent of claim 50, wherein the drug is GM-CSF.
